(19) United States
(12) Patent Application Publication
(10) Pub. No.: US 2011/0196414 A1
(43) Pub. Date: Aug. 11, 2011

(54) MULTIMODE OCCLUSION AND STENOSIS TREATMENT APPARATUS AND METHOD OF USE

(76) Inventors: Stephen Porter, Oakland, CA (US); Del Kjos, Pleasanton, CA (US); Nancy Ma, Elk Grove, CA (US)

(21) Appl. No.: 13/020,675

(22) Filed: Feb. 3, 2011

Related U.S. Application Data
(60) Provisional application No. 61/301,986, filed on Feb. 5, 2010.

(51) Int. Cl. A61F 2/01

(52) U.S. Cl. 606/200

(57) ABSTRACT

A multimode occlusion and stenosis treatment apparatus comprises an elongated member having a distal region, and an enclosure secured to the distal region of the elongated member, the enclosure comprising a flow restoring segment, an open segment distal of the flow restoring segment, and a capture segment distal the open segment. In use, a catheter is inserted into a selected blood vessel until a distal end of the catheter is distal of an occlusive or stenotic lesion in the blood vessel. The multimode occlusion and stenosis treatment apparatus is inserted into the catheter, the flow restoring segment is aligned with the lesion, and the catheter is withdrawn relative to the apparatus until a distal end of the catheter is proximal of the flow restoring segment to thereby allow the flow restoring segment to expand radially and compress the lesion against an inner surface of the blood vessel.

### RELATED APPLICATION DATA

The present application claims the benefit under 35 U.S.C. §119 to U.S. provisional patent application Ser. No. 61/301,986, filed Feb. 5, 2010. The foregoing application is hereby incorporated by reference into the present application in its entirety.

### FIELD

The field of the disclosed inventions generally relates to apparatus and methods for treating acute ischemic stroke or stenosis in a vessel of a human or veterinary patient. More particularly, the disclosed inventions relate to endovascular apparatus and methods of their use for treating vascular occlusion or stenosis.

### BACKGROUND

Vascular occlusion and stenosis significantly contribute to mortality and morbidity in patients by causing myocardial infarction and stroke. Blood vessels can become occluded (blocked) or stenotic (narrowed) in one of a number of ways. For instance, a stenosis may be formed by an atheroma or plaque, which, depending on the progression of vascular disease, may include cholesterol crystals (LDL), macrophages, and calcification deposited on the lumen walls of the blood vessel. Also, the stenosis can be induced by an embolus, or blood clot, occluding the lumen of the artery. Emboli are often formed in the chambers of the heart where they may build up in a nidus or low flow region over time. The emboli may subsequently loosen and travel distally causing a sudden obstruction in the neurocirculation. This sudden onset is often the most common cause of a deleterious acute ischemic event, where there is diminished or complete cessation of blood flow and oxygen supply to the brain. A thrombus, or blood clot formed from and adherent to an underlying atheroma, results as the atheroma builds up over time and increases plaque instability, which may resolve upon ulceration of the lesion. Plaque rupture triggers a release of clotting factors that induces thrombus formation over the unstable atheroma. This overlying thrombus may result in stenosis or complete obstruction of the artery, and or may break off at a later time and travel distally as a free embolus causing a second Ischemic event. A thrombus is typically harder than an embolus due to calcified deposits in the in the former. Rupture and re-rupture events may incorporate a sequential layering of the thrombus with the underlying atheroma resulting in increased compaction and additional hardening over time. While an embolus is typically softer than a thrombus, it can nonetheless restrict or cause a complete cessation of blood flow in the lumen of the vessel and is the most common cause of acute ischemic events.

Two different procedures have been developed to treat occlusive and stenotic lesions (“lesions”) in the vasculature. The first is to deform the lesion to reduce the restriction within the lumen of the blood vessel. This type of deformation (or dilatation) is typically performed using balloon angioplasty. Another method of treating occluded and stenotic vasculature is to attempt to completely remove either the entire lesion, or enough of the lesion to relieve the restriction in the bloods vessel. Removal of the lesion has been done through the use of radio frequency (RF) signals transmitted via conductors and through the use of lasers, both of which treatments are meant to ablate (i.e., super heat and vaporize) the lesion. Removal of the lesion has also been accomplished using aspiration, thrombectomy, or atherectomy. During thrombectomy and atherectomy, the lesion is mechanically cut into pieces or abraded away from the vessel.

Certain problems may be encountered during thrombectomy and atherectomy. The debris that is separated from the lesion is free to flow within the lumen of the vessel. If the debris flows distally, it can occlude distal vasculature and cause significant problems, including ischemic stroke from occlusion of cerebral arteries. If the debris flows proximally, it can enter another vessel and form a clot/occlusion in a previously unaffected area. This debris could lodge in the cerebral vasculature causing a stroke or in the lungs, causing a pulmonary embolism. Both of these lesion debris related diseases are highly undesirable. Angioplasty may also result in release of debris.

Prior attempts to deal with the debris or fragments have included cutting the debris into such small pieces (having a size on the order of a blood cell) that they will not occlude vessels within the problems. It is difficult to control the size of the fragments of the lesion that are severed, and larger fragments can be severed accidentally. Also, since thrombus is much softer than an atheroma, it tends to break up easier when mechanically engaged by a cutting instrument. Thus, as soon as the thrombus is mechanically engaged, there is a danger that the thrombus may be dislodged in large fragments that could occlude the vasculature.

Another attempt to deal with debris severed from a lesion is to remove the debris as it is severed using suction. However, it may be necessary to pull a relatively high vacuum in order to remove all of the pieces severed from the lesion, which can cause the vasculature to collapse. Yet another technique for dealing with debris severed from a lesion is to place a device distal to the lesion during atherectomy to catch the pieces of the lesion as they are severed, and to remove those pieces along with the capturing device when the thrombectomy or atherectomy procedure is complete. Such capture/filter devices have included expandable filters, which are placed distal of the lesion to capture lesion fragments. Such devices have also been used to capture lesion fragments that are released during angioplasty.

By way of example, such capture/filter devices are described in U.S. Pat. No. 6,129,739 to Khosravi which is incorporated herein by reference. Perceived problems with current capture devices include movement of the capture device as the thrombectomy, atherectomy, or angioplasty devices are introduced into, manipulated in, and removed from the vessel. Such movement may lead to improper positioning of the capture device and distal leakage of lesion fragments. Another perceived problem with current capture devices is the need to precisely position the capture device and thrombectomy, atherectomy, or angioplasty devices relative to each other and the lesion.

### SUMMARY

In accordance with one embodiment of the inventions disclosed and described herein, a multimode occlusion and stenosis treatment apparatus comprises an elongated member having a distal region, and an enclosure secured to the distal region of the elongated member, the enclosure comprising a flow restoring segment, an open segment distal of the flow restoring segment, and a capture segment distal of the open segment. In use, a catheter is inserted into a selected blood vessel until a distal end of the catheter is distal of a lesion in the blood vessel. The multimode occlusion and stenosis treatment apparatus is inserted into the catheter, the flow restoring segment is aligned with the lesion, and the catheter is withdrawn relative to the apparatus until a distal end of the catheter is proximal of the flow restoring segment to thereby allow the flow restoring segment to expand radially and compress the lesion against an inner surface of the blood vessel.

In another embodiment of the multimode occlusion and stenosis treatment apparatus, the elongated member is configured to be slidably disposed through a delivery catheter and has an atraumatic flexible distal tip. In yet another embodiment, the distal tip is steerable. In one embodiment, the treatment apparatus has a proximal collar connecting a proximal end of the enclosure to the elongated member, and a distal collar connecting a distal end of the enclosure to the elongated member.

In embodiments of the multimode occlusion and stenosis treatment apparatus, the flow restoring segment of the enclosure has a moderate to high cell density, the open segment of the enclosure has a low cell density, and the capture segment of the enclosure has a high cell density. The respective flow restoring segment, the open segment, and capture segment may be separate components or integrally formed. The enclosure is preferably radially compressible along a longitudinal axis and has a predetermined size when not radially compressed, wherein the flow restoring segment is radially compressible substantially independent of the capture segment. In one embodiment, the enclosure is formed from shape memory alloy, such as Nitinol. In an alternative embodiment, the flow restoring segment of the enclosure has an alternating low and moderate cell density pattern.

The capture segment is preferably configured to form a seal against an inner surface of an occluded or stenotic vessel, the cell segment having (in preferred embodiments) a cell size in a range of 20 microns to 750 microns in diameter. In one embodiment, the flow restoring segment is configured for performing an angioplasty procedure, whereas the respective open segment and capture segment are configured for performing thrombectomy and atherectomy procedures.

In accordance with a further aspect of the disclosed inventions, a method of treating vascular occlusion and stenosis is disclosed, the method comprising inserting a catheter into a selected blood vessel until a distal end of the catheter is distal of a lesion in the blood vessel, and then inserting a multimode occlusion and stenosis treatment apparatus into the catheter, the apparatus comprising an elongated member, and a compressed enclosure secured to a distal region of the elongated member, the compressed enclosure comprising a flow restoring segment, an open segment distal of the flow restoring segment, and a capture segment distal of the open segment. The method further comprises aligning the flow restoring segment with the lesion; and then withdrawing the catheter relative to the apparatus until the distal end of the catheter is proximal of the flow restoring segment to thereby allow the flow restoring segment to expand radially and compress the lesion against an inner surface of the blood vessel.

In one embodiment, withdrawing the catheter proximally relative to the apparatus allows the capture segment to expand radially and seal substantially against the inner surface of the vessel. In one embodiment, the method further includes capturing emboli in the expanded capture segment of the enclosure by advancing the catheter distally relative to the apparatus to align the distal end of the catheter with a distal end of the flow restoring segment to thereby radially compress the flow restoring segment, withdrawing the catheter and apparatus proximally to allow the lesion to pass through an opening in the open segment, and capturing the lesion in an interior of the capture segment.

Other and further aspects and features of embodiments of the disclosed inventions will become apparent from the ensuing detailed description in view of the accompanying figures.

### BRIEF DESCRIPTION OF THE DRAWINGS

Referring now to the drawings in which like reference numbers represent corresponding parts throughout, and in which:

FIG. 1 is a perspective view of a multimode occlusion and stenosis treatment apparatus constructed according to one embodiment.

FIGS. 2A-2E are schematic views illustrating various steps carried out in a treatment of vascular occlusion or stenosis using the multimode occlusion and stenosis treatment apparatus depicted in FIG. 1.

FIG. 3 is a perspective view of a multimode occlusion and stenosis treatment apparatus constructed according to another embodiment.

FIG. 4 is a perspective view of a multimode occlusion and stenosis treatment apparatus constructed according to yet another embodiment.

### DETAILED DESCRIPTION OF THE ILLUSTRATED EMBODIMENTS

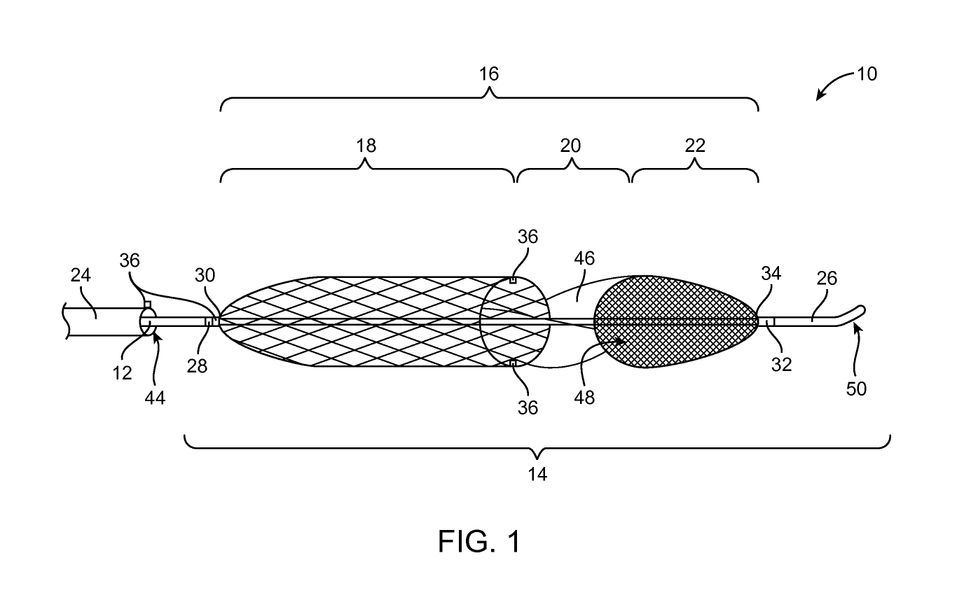

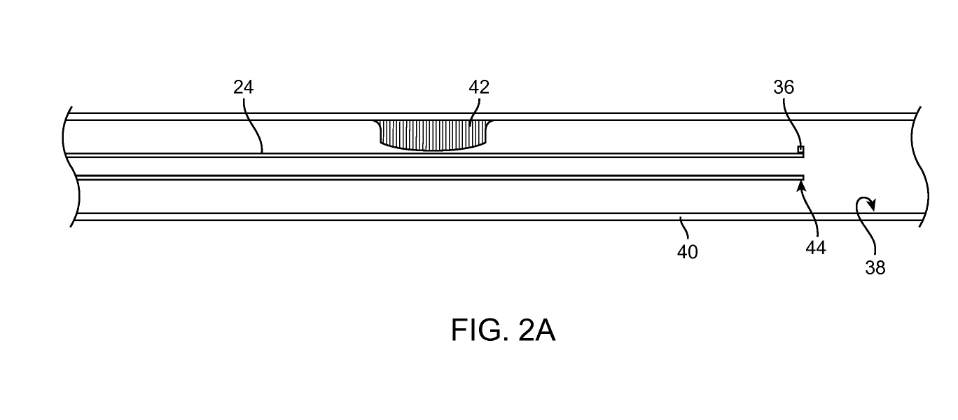

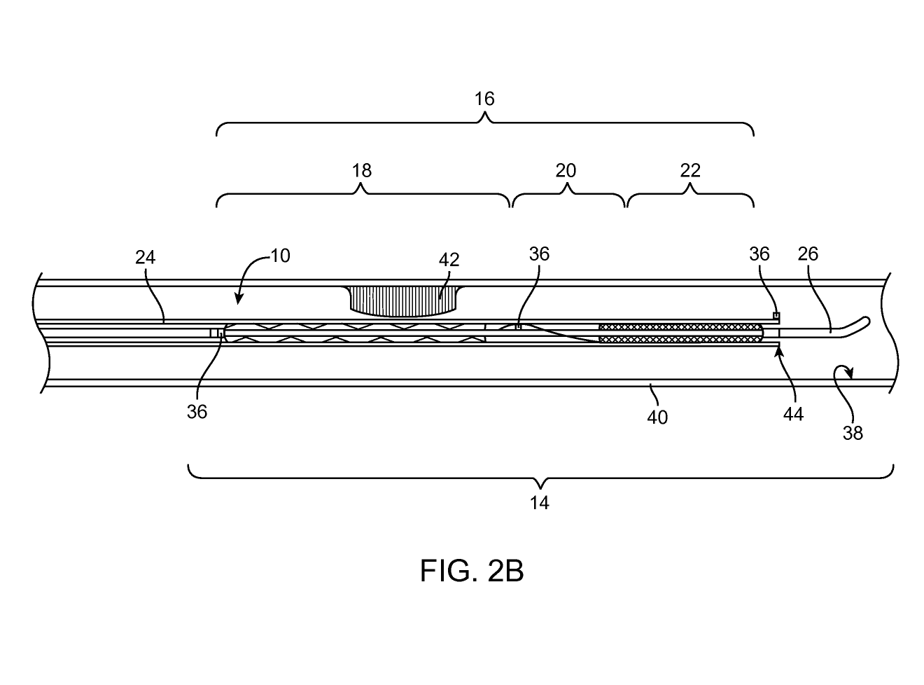

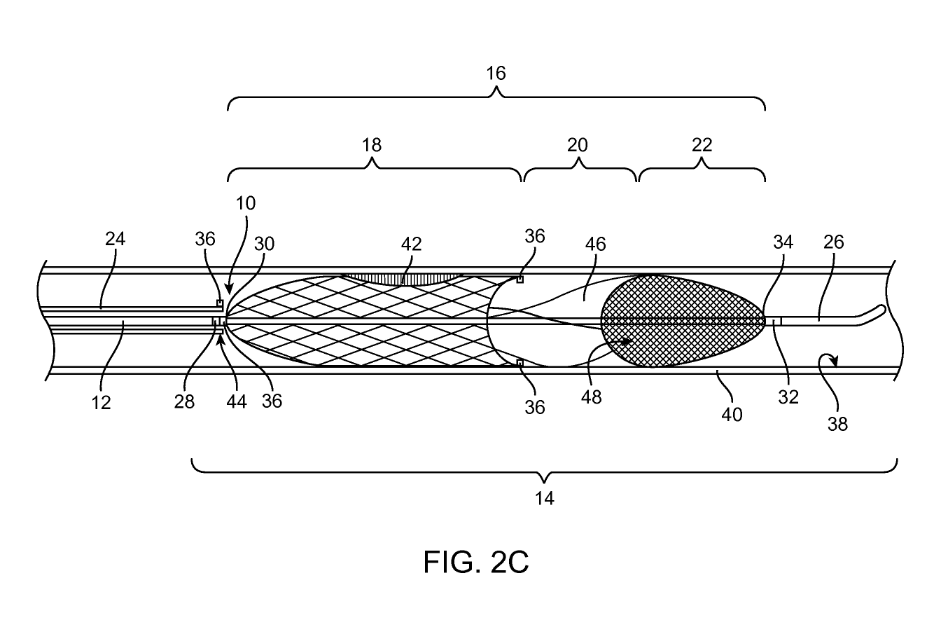

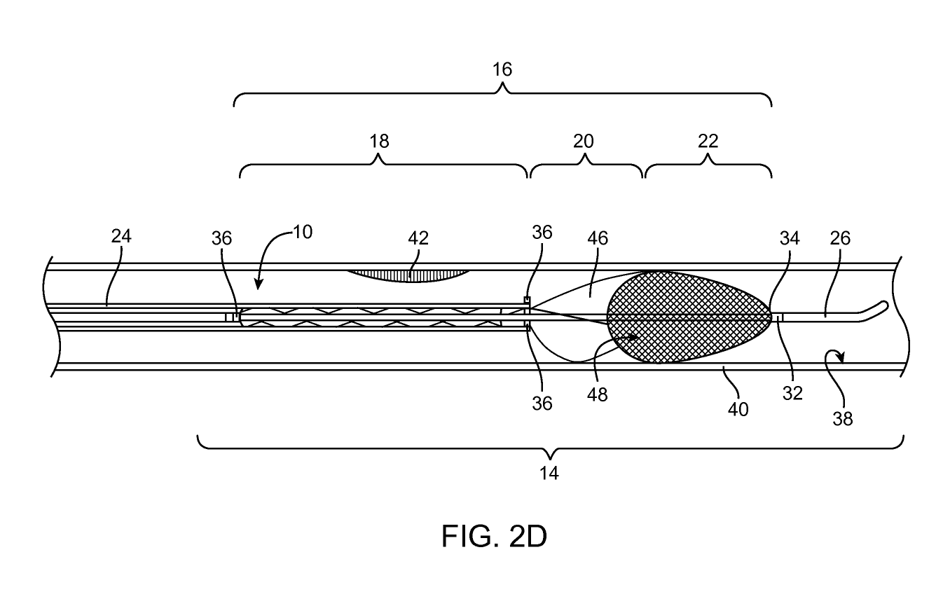

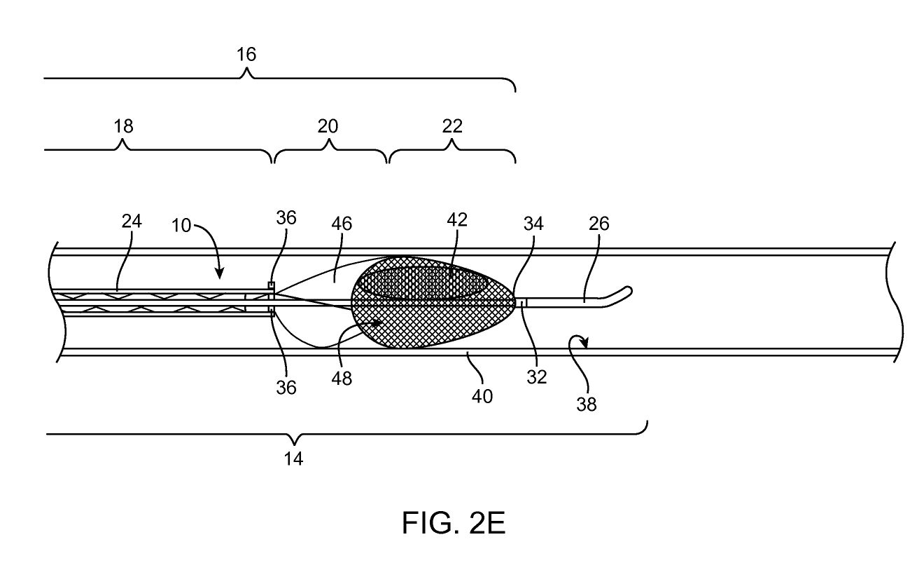

FIG. 1 illustrates an embodiment of a multimode transluminal occlusion and stenosis treatment apparatus 10. The apparatus 10 includes an elongated member 12, which has a distal region 14. An enclosure 16 is mounted on the distal region 14 of the elongated member 12. The enclosure is configured to treat a lesion 42 in a vessel 40 of a patient (see FIGS. 2A-2E). The apparatus 10 also includes a catheter 24, in which the elongated member 12 and enclosure 16, in its compressed profile, may be slidably disposed.

The elongated member 12 may be a guide wire of sufficient strength and stiffness to negotiate the vasculature of a patient from an introduction site to the lesion 42. Alternatively, the elongated member 12 may be a tube of sufficient strength and stiffness. The elongated member 12 may be formed from stainless steel. When the apparatus 10 is in use, the proximal end (not shown) of the elongated member 12 extends from the introduction site to allow a user to manipulate elongated member 12 and the enclosure 16 mounted thereto. A steerable distal tip 26 is mounted at the distal end 50 of the elongated member 12. The steerable distal tip 26 can be operated using known mechanisms for guiding the elongated member 12 through a patient's vasculature.

The enclosure 16 is connected to the distal region 14 of the elongated member 12 by a proximal collar 28 at the proximal end 30 of the enclosure 16 and by a distal collar 32 at the distal end 34 of the enclosure 16. The enclosure 16, the collars 28, 32, and the elongated member 12 may be joined by known methods including spot welding and the use of adhesives. Alternatively, one of the collars, e.g. the distal collar 32, may be joined to the enclosure 16, but slidably mounted on the elongated member 12. Such a construction allows the enclosure 16 to lengthen distally as it is compressed radially.

The enclosure 16 is integrally formed to minimize the number of parts in the apparatus 10. The enclosure 16 may be woven from wire with the cell density and cell size determined by the pattern and density of the weave. Weaving the enclosure 16 from an alloy with shape memory, like Nitinol, allows the enclosure 16 to be compressed radially for introduction through the vasculature while returning to a predetermined configuration and size when the compressive force is removed. Different segments of the enclosure 16 can be formed by varying the pattern and density of the weave.

The enclosure 16 is divided into three structurally and functionally distinct segments. A flow restoring segment 18 is located at the proximal end 30 of the enclosure 16. The flow restoring segment 18 has a moderate to high cell density and moderate to small cell size, i.e. about 5 to about 10 cells per circumferential section. In its expanded profile, the flow restoring segment 18 is generally tube shaped. At the proximal end 30 of the enclosure 16, the flow restoring segment 18 tapers to a cone as it connects to the proximal collar 28. The distal end of the flow restoring segment is open. The flow restoring segment 18 is configured to compress a lesion 42 radially against the inner surface 38 of the vessel 40, as shown in FIG. 2C.

An open segment 20 is located distal of the flow restoring segment 18 and in the middle of the enclosure 16. The open segment 20 has a low cell density, i.e. about 2 to about 5 cells per circumferential section and large cell size, i.e. about 2 mm to about 6 mm in longitudinal length. The open segment 20 has very few wires (i.e., about 2 to about 5 wires) that approximate a tube shape with open proximal and distal ends in its expanded profile. The cells of the open segment 20 form openings 46 that allow lesions 42 to penetrate the enclosure 16 and enter the interior 48 of the capture segment 22.

A capture segment 22 is located distal of the open segment 20 and at the distal end 34 of the enclosure 16. The capture segment 22 has a high cell density and a small cell size, i.e. about 6 to about 25 cells per circumferential section. The capture segment 22 is generally cone shaped. The capture segment 22 is open at the proximal end and tapers as it connects to the distal collar 32 at the distal end 34 of the enclosure 16. At its open proximal end, the capture segment 22 approximates the cross sectional shape and size of the vessel 40 and substantially seals against the inner surface 38 of the vessel 40 when expanded. The capture segment 22 is configured to filter emboli and to mechanically cut or abrade the lesion 42 from the inner surface 38 of the vessel 40.

The enclosure 16 is capsule shaped and tapers at the proximal and distal ends 30, 34 to connect to the proximal and distal collars 28, 32, respectively. Because of its moderate to high cell density and moderate to small cell size, a compressed flow restoring segment 18 in an occluded or stenotic vessel expands radially and increases the lumen cross sectional area of the vessel in an angioplasty procedure, as shown in FIGS. 2B and 2C. The openings 46 in the open segment 20 allow access to the interior of the enclosure 16, including to the interior 48 of the filter segment 22. During the angioplasty, debris or emboli flow downstream and enter the enclosure 16 through the openings 46 in the open segment 20. The debris and emboli are then captured in the interior 48 of the capture segment 22. The enclosure 16 is formed so that the relative position of the flow restoring segment 18 and the capture segment 22 maximizes capture of debris and emboli. Because it substantially seals against the inner surface 38 of the vessel 40 when expanded, the capture segment 22 can also mechanically remove a lesion 42 from the inner surface 38 and a vessel 40 as the capture segment 22 is pulled proximally past the lesion 42 in an atherectomy or thrombectomy procedure, as shown in FIGS. 2D and 2E.

In order to allow red blood cells (5 micron in diameter) to pass easily through the capture segment 22, the weave pattern and density of the capture segment 22 generate cell sizes in a range of about 20 to about 750 microns in diameter. This size restricts flow of debris or emboli while allowing free flow of red blood cells. Due to the low number of cells and wires in the open segment 20, the flow restoring segment 18 is compressible substantially independent of the capture segment 22, as shown in FIGS. 2D and 2E. In some embodiments, radiopaque markers 36 are secured to the proximal and distal ends of the flow restoring segment 18 to allow fluoroscopic positioning of the flow restoring segment 18 adjacent to a lesion 42.

The flow restoring segment 18 is configured to primarily provide a blood flow by-pass to quickly restore blood flow to ischemic regions of the brain and mitigate the impact of prolonged ischemia. Because this may not provide permanent treatment, a means for dislodging and capturing the lesion 42 is provided with the open segment 20 and capture segment 22. The open segment 20 is configured to allow passage of a lesion 42 into the interior of the enclosure 16 upon withdrawal of the apparatus 10 either while fully expanded or partially re-sheathed (where the flow restoration segment 18 is re-sheathed and the remaining segments 20, 22 are used to capture the lesion 42 and debris.)

While various enclosures 16 have been shown and described, they have been presented for purposes of illustration, and not limitation, of the disclosed inventions. One of skill in the art will appreciate that various modifications may be made to the enclosures 16. In alternative embodiments, the enclosure 16 may include more than one flow restoring segment 18, more than one open segment 20, and/or more than one capture segment 22.

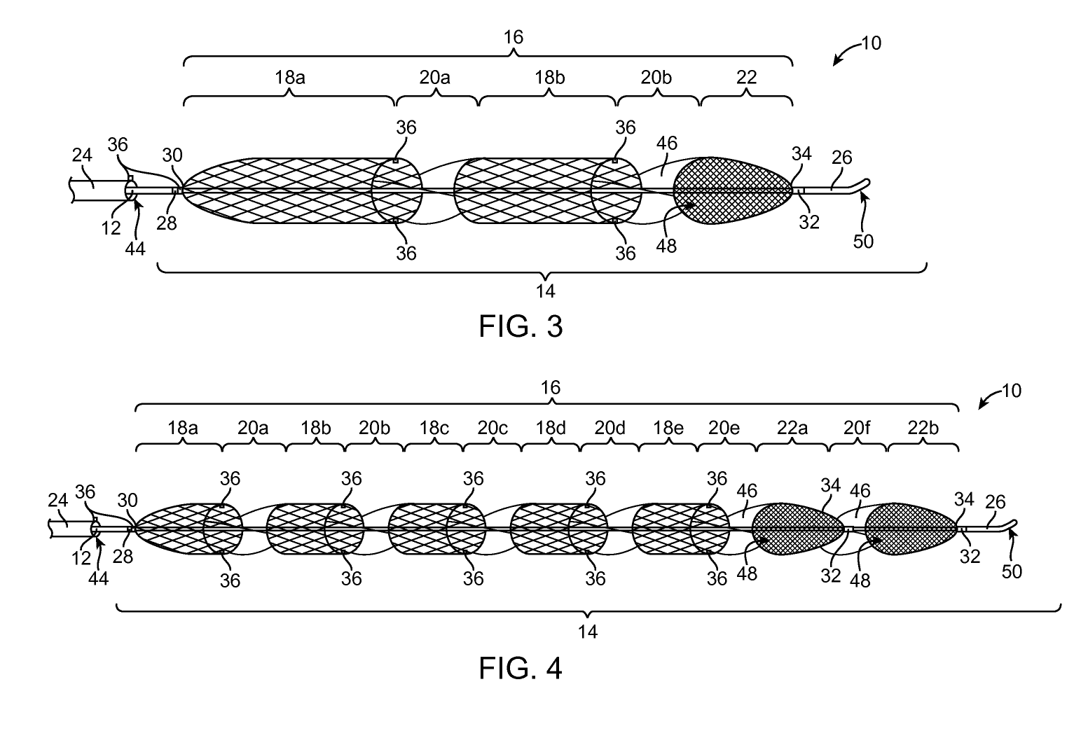

By way of non-limiting example, the enclosure 16 in FIG. 3 includes a first flow restoring segment 18a followed distally by a first open segment 20a, then a second flow restoring segment 18b, a second open segment 20b, and finally a capture segment 22. Also by way of non-limiting example, the enclosure 16 in FIG. 4 includes a first flow restoring segment 18a followed distally by a first open segment 20a, then four iterations of a flow restoring segment followed by an open segment (18b, 20b, 18c, 20c, 18d, 20d, 18e, 20e), a first capture segment 22a, a sixth open segment 20f, and finally a second capture segment 22b.

The catheter 24 is generally tubular and extends through the vasculature of a patient from an introduction site to the lesion 42. In use, the proximal end (not shown) of the catheter 24 extends from the introduction site to allow a user to manipulate the catheter 24. The catheter 24 is sized to be threaded past the lesion 42 and to the carry the elongated member 12 and the enclosure 16 in its compressed profile. Lubricious coatings, such as Teflon®, can be applied to the inner and outer surfaces of the catheter 24 to facilitate insertion of the catheter 24 through the vasculature and insertion of the elongated member 12 and the enclosure 16 through the catheter 24. In some embodiments, radiopaque markers 36 are secured to the distal end 44 of the catheter 24 to allow fluoroscopic positioning of the distal end 44 of the catheter 24 relative to a lesion 42 and the enclosure 16.

FIGS. 2A to 2E illustrate the treatment of vascular occlusion or stenosis using the multimode occlusion and stenosis treatment apparatus 10. In FIG. 2A, the vessel 40 is shown with a significant blockage caused by a lesion 42 attached to the inner surface 38 of the vessel 40. While the exemplary lesion 42 shown in FIG. 2A is attached to only one wall of the vessel 40, the apparatus and method can be used to treat other types of lesions, including ring shaped lesions that overlay the entire inner surface 38 of a cross section of the vessel 40.

In FIG. 2A, a catheter 24 is inserted into the vessel 40 through an introduction site until the distal end 44 of the catheter 24 is distal of the lesion 42. The lubricious coating on the outer surface of the catheter 24 reduces frictional resistance during the insertion. The distal end 44 of the catheter 24 is positioned distal of the lesion 42 by a distance approximately equal to the length of the enclosure 16 in its compressed profile. As described above, the relative position of the distal end 44 of the catheter 24 and the lesion 42 can be monitored fluoroscopically.

In FIG. 2B, the catheter 24 is held stationary as an elongated member 12 and the enclosure 16 attached therein (in its compressed profile) is threaded through the proximal end of the catheter 24 to the lesion 42. The lubricious coating on the inner surface 38 of the catheter 24 reduces frictional resistance as the elongated member 12 and the enclosure 16 are threaded through the catheter 24. The steerable distal tip 26 helps the respective elongated member 12 and enclosure 16 be navigated through the tortuous vasculature. Using fluoroscopy aided by radiopaque markers 36 secured to the catheter 24 and the enclosure 16, the elongated member 12 and the enclosure 16 are positioned within the catheter 24 such that the entire enclosure 16 remains compressed inside of the catheter 24 while the steerable distal tip 26 extends out the distal end 44 of the catheter 24. The catheter 24 and the elongated member 12 and enclosure 16 contained therein are positioned within the vessel 40 to align the flow restoring segment 18 with the lesion 42.

In FIG. 2C, the elongated member 12 and the enclosure 16 are held stationary relative to the vessel 40 using the proximal end of the elongated member 12 as the catheter is withdrawn proximally until the distal end 44 of the catheter 24 is proximal of the proximal end 30 of the enclosure 16. The relative positions are determined by fluoroscopy. Because the enclosure is formed of shaped memory alloy like Nitinol, once the compressive force of the catheter 24 is removed, the enclosure 16 expands radially to its expanded profile. The relatively elastic material provides sufficient resilient force so that the flow restoring segment 18 compresses the lesion 42 and the capture segment 22 substantially seals against the inner surface 38 of the vessel 40 to capture any debris or emboli produced during this angioplasty procedure. As the debris and emboli are generated at the lesion 42, they enter the enclosure through the flow restoring segment 18 and the open segment 20 to be captured in the interior 48 of the capture segment 22.

In FIG. 2D, the elongated member 12 and the enclosure 16 are held stationary relative to the vessel 40 using the proximal end of the elongated member 12 as the catheter is advanced distally until the distal end 44 of the catheter 24 is just proximal of the flow restoring segment 18 as determined by fluoroscopy. The relative distal movement of the catheter 24 over the flow restoring segment 18 of the enclosure 16 re-sheaths the flow restoring segment 18 by radially compressing back into its compressed profile. Because the flow restoring segment 18 is compressible substantially independent of the capture segment 22, debris or emboli that may be generated during the re-sheathing are captured by the distally located and fully expanded capture segment 22. After the flow restoring segment 18 is re-sheathed, a compressed lesion 42 remains attached to the inner surface 38 of the vessel 40.

In FIG. 2E, the catheter 24, elongated member 12, and enclosure 16, are held stationary relative to each other and all three are simultaneously withdrawn proximally out of the vessel 40. As the open segment 20 passes over the compressed lesion 42, the lesion 42 enters the enclosure 16 through openings 46 in the open segment 20. As the expanded capture segment 22 passes over the compressed lesion 42, the capture segment 22 mechanically cuts or abrades the compressed lesion 42 from the inner surface 38 and the vessel 40. The severed lesion 42 is then captured in the interior 46 of the capture segment 22 and removed from the vessel 40 along with the catheter 24, elongated member 12, and enclosure 16.

While various embodiments of the disclosed inventions have been shown and described, they are presented for purposes of illustration, and not limitation. It will be appreciated that various modifications may be made to the illustrated and described embodiments without departing from the scope of the disclosed inventions, which is to be defined only by the following claims and their equivalents.

What is claimed is:

1. A multimode occlusion and stenosis treatment apparatus, comprising:
an elongated member having a distal region; and
an enclosure secured to the distal region of the elongated member, the enclosure comprising
a flow restoring segment,
an open segment distal of the flow restoring segment, and
a capture segment distal the open segment.

2. The treatment apparatus of claim 1, further comprising a catheter, wherein the elongated member is slidably disposed in the catheter, the elongated member comprising an atraumatic flexible distal tip.

3. The treatment apparatus of claim 1, further comprising a proximal collar connecting a proximal end of the enclosure to the elongated member, and a distal collar connecting a distal end of the enclosure to the elongated member.

4. The treatment apparatus of claim 1, wherein the flow restoring segment of the enclosure has a moderate to high cell density, the open segment of the enclosure has a low cell density, and the capture segment of the enclosure has a high cell density.

5. The treatment apparatus of claim 1, wherein the respective flow restoring segment, the open segment, and capture segment are integrally formed.

6. The treatment apparatus of claim 1, wherein the enclosure is radially compressible along a longitudinal axis and has a predetermined size when not radially compressed.

7. The treatment apparatus of claim 6, wherein the flow restoring segment is radially compressible substantially independent of the capture segment.

8. The apparatus of claim 1, wherein the enclosure comprises a shape memory alloy.

9. The apparatus of claim 1, wherein the capture segment is configured to form a seal against an inner surface of a vessel.

10. The apparatus of claim 1, wherein the capture segment has a cell size in a range of 20 microns to 750 microns in diameter.

11. The apparatus of claim 1, wherein the flow restoring segment is configured for performing an angioplasty procedure, and wherein the respective open segment and capture segment are configured for performing thrombectomy and atherectomy procedures.

12. The apparatus of claim 1, the enclosure further comprising one or more additional flow restoring segments.

13. The apparatus of claim 1, the enclosure further comprising one or more additional open segments.

14. The apparatus of claim 1, the enclosure further comprising one or more additional capture segments.

15. A multimode occlusion and stenosis treatment apparatus, comprising:
a catheter,
an elongated member slidably disposed in the catheter and having a distal region; and
an enclosure secured to the distal region of the elongated member, the enclosure comprising
a flow restoring segment having a moderate to high cell density,
an open segment distal of the flow restoring segment and having a low cell density, and
a capture segment distal the open segment and having a high cell density.

16. The treatment apparatus of claim 15, wherein the enclosure is radially compressible along a longitudinal axis and has a predetermined size when not radially compressed, wherein the flow restoring segment is radially compressible substantially independent of the capture segment.

17. A method of treating vascular occlusion or stenosis, comprising:
inserting a catheter into a selected blood vessel until a distal end of the catheter is positioned distal of an occlusive or stenotic lesion in the blood vessel;
inserting a multimode occlusion and stenosis treatment apparatus into the catheter, the apparatus comprising
an elongated member, and
a compressed enclosure secured to a distal region of the elongated member, the compressed enclosure comprising
a flow restoring segment,
an open segment distal of the flow restoring segment, and
a capture segment distal of the open segment;
aligning the flow restoring segment with the lesion; and
withdrawing the catheter relative to the apparatus until the distal end of the catheter is proximal of the flow restoring segment to thereby allow the flow restoring segment to expand radially and compress the lesion against an inner surface of the blood vessel.

18. The method of claim 17, wherein withdrawing the catheter proximally relative to the apparatus allows the capture segment to expand radially and seal substantially against the inner surface of the vessel.

19. The method of claim 18, further comprising capturing emboli in the expanded capture segment of the enclosure.

20. The method of claim 19, further comprising:
advancing the catheter distally relative to the apparatus to align the distal end of the catheter with a distal end of the flow restoring segment to thereby radially compress the flow restoring segment;
withdrawing the catheter and apparatus proximally to allow the lesion to pass through an opening in the open segment; and
capturing the lesion in an interior of the capture segment.
